(19) United States
(12) Patent Application Publication
(10) Pub. No.: US 2026/0108235 A1
(43) Pub. Date: Apr. 23, 2026

(54) FLUID ASSESSMENT FOR DETERMINING QUALITY OF LOWER GASTROINTESTINAL TRACT PREPARATION PRIOR TO MEDICAL, SURGICAL, AND ENDOSCOPIC PROCEDURES

(71) Applicant: PAISLEY IP LLC, Independence, MO (US)

(72) Inventor: Florence M. Hosseini-Aslinia, Leawood, KS (US)

(73) Assignee: PAISLEY IP LLC, Independence, MO (US)

(21) Appl. No.: 19/344,288

(22) Filed: Sep. 29, 2025

Related U.S. Application Data
(60) Provisional application No. 63/700,234, filed on Sep. 27, 2024.

(51) Int. Cl. A61B 10/00; G06T 7/00

(52) U.S. Cl. CPC A61B 10/0038; G06T 7/0012; G06T 2207/20081

(57) ABSTRACT

Systems and methods for objective evaluation of a stool sample to determine whether a patient's lower gastrointestinal tract is sufficiently clean to undergo a successful gastrointestinal visualization procedure with favorable visualization. A method includes collecting a stool sample from a patient and assessing the stool sample to determine whether an intestine of the patient is sufficiently clean for the patient to undergo a successful lower gastrointestinal tract visualization procedure.

### CROSS-REFERENCE TO RELATED APPLICATIONS

This Application claims the benefit of U.S. Provisional Patent Application No. 63/700,234, filed Sep. 27, 2024 titled “GASTROINTESTINAL TRACT PREPARATION PRIOR TO MEDICAL, SURGICAL, AND ENDOSCOPIC PROCEDURES,” which is incorporated herein by reference in its entirety, including but not limited to those portions that specifically appear hereinafter, the incorporation by reference being made with the following exception: In the event that any portion of the above-referenced applications are inconsistent with this application, this application supersedes the above-referenced applications.

### TECHNICAL FIELD

The disclosure relates generally to systems, methods, and devices for assessing a fluid sample, and particularly relates to systems, methods, and devices for assessing a stool sample to determine whether a patient is sufficiently prepared to undergo a successful gastrointestinal procedure.

### BACKGROUND

Colorectal cancer (CRC) is the third most common cancer in the United States and the second leading cause of cancer-related deaths worldwide. CRC often begins with benign precancerous polyps in the colon, and these precancerous polyps may eventually develop into cancerous growths. The risk of CRC is significantly reduced when patients undergo early detection procedures, and specifically when patients receive regular colonoscopy screenings. The colonoscopy procedure provides a comprehensive examination of the patient's colon, and this enables physicians to excise precancerous polyps or growths.

The Centers of Disease Control in the United States reports a steady decline in incidences of CRC since adults aged 50 years and older were recommended to receive regular colonoscopy screenings. In the United States, studies indicate that regular colonoscopy screenings reduce the incidence of CRC by 83% and reduce the risk of mortality associated with CRC by 89%.

However, colonoscopy procedures are fallible, and the effectiveness of a colonoscopy is highly dependent on the patient's preparation for the colonoscopy and whether the colon is sufficiently clean to allow comprehensive visualization of the colon. Research using tandem colonoscopies, wherein two independent colonoscopy providers examined the same patient on the same day in a research setting, have found that approximately 25% of polyps may be missed in a single colonoscopy. It can be important to ensure proper bowel preparation to improve polyp detection and advance CRC prevention efforts. Currently, up to 30% of colonoscopy procedures are conducted on colons that lack optimal cleanliness. This increases the risk that a provider misses one or more polyps within the patient's colon and ultimately fails to detect the presence of CRC.

Currently, there is no objective measure to determine the quality of bowel preparation prior to undergoing a gastrointestinal endoscopic procedure like a colonoscopy. The currently accepted procedure is to instruct patients to visually examine bowel output prior to the procedure and make a best guess on whether the bowel is sufficiently clean. This increases the risk of the provider being unable to identify polyps and early cancer, and thus may eliminate the benefit of performing the colonoscopy procedure.

What is needed are systems, methods, and devices to objectively and accurately determine whether a patient has sufficiently prepared for a lower gastrointestinal procedure by cleansing the lower gastrointestinal tract. In light of the foregoing, disclosed herein are systems, methods, and devices for assessing a fluid sample to objectively determine whether a lower gastrointestinal tract of a patient is sufficiently prepared for a lower gastrointestinal endoscopic procedure such as a colonoscopy.

### BRIEF DESCRIPTION OF THE DRAWINGS

Non-limiting and non-exhaustive implementations of the present disclosure are described with reference to the following figures, wherein like reference numerals refer to like parts throughout the various views unless otherwise specified. Advantages of the present disclosure will become better understood with regard to the following description and accompanying drawings where:

FIG. 1 is a schematic block diagram of a method for determining whether a gastrointestinal tract of a patient is sufficiently clean to undergo a gastrointestinal procedure with favorable visualization;

FIG. 2 is a schematic illustration of a perspective view of a system for capturing and evaluating a stool sample to determine whether a lower gastrointestinal tract is sufficiently clean to allow favorable visualization in preparation for a colonoscopy procedure;

FIG. 3 is a perspective view of a Secchi disk;

FIG. 4A is a schematic illustration of a perspective view of a system for evaluating a stool sample to determine whether a lower gastrointestinal tract is sufficiently clean to allow favorable visualization in preparation for a colonoscopy procedure;

FIG. 4B is a schematic illustration of a straight-on cross-sectional view of a system for evaluating a stool sample to determine whether a lower gastrointestinal tract is sufficiently clean to allow favorable visualization in preparation for a colonoscopy procedure;

FIG. 5A is a schematic illustration of a perspective view of a system for evaluating a stool sample to determine whether a lower gastrointestinal tract is sufficiently clean to allow favorable visualization in preparation for a colonoscopy procedure;

FIG. 5B is a schematic illustration of a straight-on cross-sectional view of a system for evaluating a stool sample to determine whether a lower gastrointestinal tract is sufficiently clean to allow favorable visualization in preparation for a colonoscopy procedure;

FIG. 6 is a schematic illustration of a system and process flow for capturing and evaluating a stool sample to determine whether a lower gastrointestinal tract is sufficiently clean to allow favorable visualization in preparation for a colonoscopy procedure;

FIG. 7 is a schematic illustration of an exploded view of components of a system for turbidimetry;

FIG. 8 is a schematic illustration of an exploded view of components of a system for spectrophotometry;

FIG. 9A is a schematic block diagram of a system and process flow for calibrating a spectrophotometer and utilizing a calibrated spectrophotometer to assess a patient stool sample;

FIG. 9B is a schematic block diagram of a system and process flow for calibrating a nephelometer and utilizing a calibrated nephelometer to assess a patient stool sample;

FIG. 9C is a schematic block diagram of a system and process flow for calibrating a turbidimeter and utilizing a calibrated turbidimeter to assess a patient stool sample;

FIG. 9D is a schematic block diagram of a system and process flow for calibrating a refractometer and utilizing a calibrated refractometer to assess a patient stool sample;

FIGS. 10A and 10B are schematic illustrations of a system 1000 for evaluating whether the lower gastrointestinal tract of a patient is sufficiently clean to undergo a successful gastrointestinal visualization procedure with favorable visualization;

FIG. 11 is a schematic illustration of a system for simultaneous nephelometry and turbidimetry for determining a turbidity of a sample;

FIG. 12 is a schematic block diagram of a system for assessing image data and sensor data, and further for facilitating patient-provider communications;

FIG. 13 is an image of a specimen collector for collecting a stool sample from a patient;

FIG. 14 is an image of a specimen collector for collecting a stool sample from a patient;

FIG. 15 is an image of a specimen collector for collecting a stool sample from a patient;

FIG. 16 is an image of a specimen collector for collecting a stool sample from a patient; and

FIG. 17 is a schematic block diagram of components of an exemplary computing system.

### DETAILED DESCRIPTION

Colorectal cancer (CRC) is the third most common cancer in the United States. CRC typically begins as small clumps of cells called polyps that form inside the colon. Polyps generally are not cancerous, but some may turn into colon cancers over time. Because polyps often do not cause any symptoms, doctors recommend regular screening tests to look for polyps in the colon. Finding and removing polyps helps prevent colon cancer, and thus, regular screenings, combined with polyp detection and removal, has significantly decreased the incidence of CRC in the United States.

CRC is best detected by way of an endoscopic imaging procedure that examines the gastrointestinal tract for abnormalities. A colonoscopy is an exam for detecting changes or abnormalities in the gastrointestinal tract. During a colonoscopy, a specialized endoscope referred to as a colonoscope is inserted into the large intestine of the patient. The colonoscope includes a long, flexible tube that may be inserted into the patient by way of the rectum. The colonoscope includes a small video camera at the distal end of the device that allows a practitioner or computer program to view the interior of the colon. Tissue samples for biopsies may also be taken during the colonoscopy. If necessary, polyps or other types of abnormal tissue can be removed with the colonoscope during the colonoscopy.

The effectiveness of a colonoscopy depends on the cleanliness of the lower gastrointestinal tract because poor preparation may hinder the physician's ability to visualize polyps. If the lower gastrointestinal tract is not clean, then it is difficult for the physician to visualize polyps with the camera on the colonoscope. Lower gastrointestinal tract cleanliness prior to a colonoscopy is currently measured subjectively, and up to one-third of colonoscopy procedures encounter suboptimal lower gastrointestinal tract cleanliness. This increases the risk of missing polyps and may therefore increase the risk of failing to detect early cancer.

What is needed are systems, methods, and devices to objectively determine lower gastrointestinal tract cleanliness prior to a colonoscopy to allow for favorable visualization during a procedure. In view of the foregoing, the systems, methods, and devices described herein are utilized to objectively determine whether the colon of a patient is sufficiently clean to provide favorable visualization during a colonoscopy procedure. If the large intestine is sufficiently clean, then the healthcare provider can view the lining of the patient's colon, identify potentially cancerous tissue within the patient's colon, and identify polyps or other precancerous tissues within the patient's colon with better precision due to increased visualization of the surgical scene. The patient is typically instructed to undergo colonoscopy preparation several days before the colonoscopy procedure.

Typically, the patient is instructed to eat a light diet for three or four days before the procedure. The patient may also be instructed to consume low-fiber foods that are easy to digest and do not typically cling to the sides of the large intestine. Low-fiber foods may include, for example, white bread, pasta, rice, well-cooked vegetables without skin, fruit without skin or seeds, lean meats, chicken, fish, eggs, and so forth. The patient may be instructed to avoid seeds, nuts, popcorn, fatty foods, tough meat, whole grains, raw vegetables, fruit with seeds or peels, corn broccoli, cabbage, beans, peas, and so forth. The patient is then instructed to fast the day before the procedure, and consume only clear liquids, such as sports drinks, clear juice, clear broth, and water. The patient is instructed to consume strong laxatives the night before the colonoscopy to clear the colon. In some cases, the patient is instructed to drink a half-gallon of liquid laxative in the evening, and then another half-gallon of liquid laxative about six hours before the colonoscopy appointment. When the laxative begins working, the patient will experience frequent diarrhea as the intestines are purged in preparation for the colonoscopy. If the colonoscopy preparation is successful, then the patient's stool should appear like urine or clear water prior to the colonoscopy.

Many patients find it difficult to assess the success of their colonoscopy preparation, when relying on subjective judgments about bowel cleanliness. A study published by the Annals of Gastroenterology that took place over a 10 year period and that included 28,725 patients cited that approximately 23% of patients had inadequate bowel preparation prior to a colonoscopy. Another study published by Translational Gastroenterol Hepatol cited that approximately 18% of colonoscopies were canceled where a major factor in cancellations was inadequate bowel preparation. Therefore, the present technology seeks to overcome the problems and difficulties associated with inadequate bowel preparation prior to a medical procedure. The systems, methods, and devices described herein are leveraged to objectively determine whether the patient is prepared for the colonoscopy or other medical procedure including whether a lower gastrointestinal tract of a patient is sufficiently prepared for the patient to undergo a successful colonoscopy procedure.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

Before the systems, methods, and devices for objectively assessing lower gastrointestinal tract cleanliness prior to a colonoscopy procedure are disclosed and described, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified ingredients, materials, or steps and those that do not materially affect the basic and novel characteristic or characteristics of the claimed disclosure.

As used herein, “effective amount” means an amount of an ingredient or a component of the product that is nontoxic, but sufficient to provide the desired effect and performance at a reasonable benefit/risk ratio attending any dietary supplement or product.

As used herein, the term “digital data capturing device” refers to a device that is capable of capturing data associated with a stool sample. The digital data capturing device may capture data that is digital or is converted to be digital. The digital data capturing device may capture an image, for example, the device may be an image capturing device such as a camera, a digital camera, a Charge Couple Device (CCD), a Complementary Metal Oxide Semiconductor (CMOS), and the like. The digital data capturing device may also be capable of capturing electromagnetic radiation, such as light, that has passed through the stool sample or reflected off of the stool sample. The digital data capturing device may be referred to as a sensor. Examples of digital data capturing devices can include spectrophotometers, nephelometers, refractometers, turbidimeters, spectrometers, microscopes, photometers, colorimeters, polarimeters, ellipsometers, and the like.

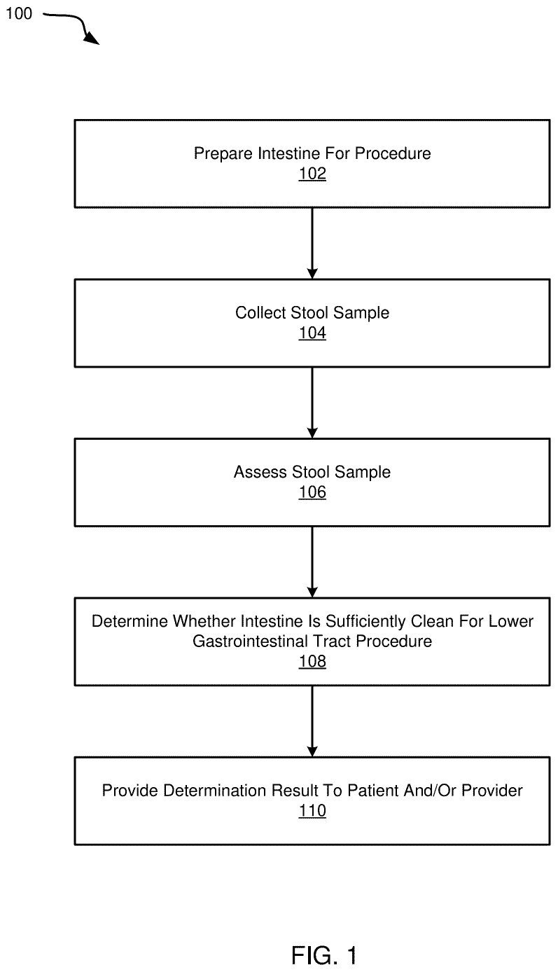

Referring now to the figures, FIG. 1 is a schematic flowchart diagram of a method 100 for determining whether a lower gastrointestinal tract is sufficiently clean to undergo a successful lower gastrointestinal tract procedure with favorable visualization. The method 100 may be utilized to determine cleanliness prior to an endoscopy procedure such as a pouchoscopy, sigmoidoscopy, colonoscopy, ileoscopy, enteroscopy, or other procedure. It will be appreciated that there is a great benefit to having a clean gastrointestinal tract because it results in better visualization of the surgical scene during the procedure.

The method 100 includes preparing at 102 the intestine for the procedure, which may specifically include an endoscopic visualization procedure such as a pouchoscopy, sigmoidoscopy, colonoscopy, ileoscopy, enteroscopy, or other procedure. The preparation at 102 may include consuming a light, low-fiber diet for three to four days. This diet may include easy-to-digest foods such as white bread, pasta, well-cooked vegetables without skin, lean meats, and eggs. This diet may be free from foods such as seeds, nuts, popcorn, raw fruits, and raw vegetables. The day before the procedure the preparation may include drinks clear liquids such as sports drinks and broths. The preparation may further include consuming strong laxatives, wherein half of he prescribed laxative may be consumed in the evening prior to the procedure, and the other half of the prescribed laxative may be consumed hours before the appointment. This may lead to frequent diarrhea. A successful preparation will result in the patient's stool comprising a clear liquid that looks similar to urine.

The method 100 includes collecting at 104 a stool sample during and after the preparation 102. The stool sample may be collected in a collection device, which may include one or more of a commode specimen collector, disposable stool collection bag, stool sample container, bedpan, fecal management system (FMS), toilet bowl, colostomy bag, or other collection device. The collection device may be a reusable device constructed of metal or another durable material that may undergo a sterilization cycle. The collection device may be a single-use device constructed of a durable material such as plastic. The collection device may be a single-use device constructed of a flushable and/or biodegradable material. The means for collecting at 104 may vary depending on the patient's circumstances, and various collection devices and systems may be utilized without departing from the scope of the disclosure.

The stool sample may be provided by the patient to a health care professional in a specimen containing device. The specimen containing device may be the collection device or the stool sample may be transferred from the collection device to the specimen containing device. The specimen containing device may include a disposable stool collection bag, a stool sample container, a bedpan, a fecal management system (FMS), a toilet bowl, a colostomy bag, and/or a bowl.

In some cases, the collecting at 104 may include several separate collection instances over a certain time period. The collecting at 104 may further include collecting multiple stool samples at one instance. In either case, the sample is collected and then each of the multiple stool samples is separately assessed. In one implementation, the patient deposits the stool sample into a container such as a toilet bowl and captures an image of the stool sample in the toilet bowl using a digital camera. The digital camera may be associated with a mobile device such as a smart phone. The mobile device may be capable of sending images over a network such as the internet. The image may be sent to the health care provider via the mobile device over a network. The image may be sent to the health care provider instead of providing the stool sample to the healthcare provider. In one implementation, the patient may download and install software, such as an app, from the healthcare provider onto the mobile device. The software may be employed with the mobile device to capture the image and send the image to the healthcare provider.

The method 100 includes assessing at 106 the stool sample. The assessment at 106 may include assessing the fluid quality of the stool sample to determine or measure the clarity of the stool sample and may specifically include measuring the turbidity of the stool sample. In one implementation, the turbidity of the stool sample is a factor in calculating or measuring the clarity of the stool sample. The turbidity of the stool sample refers to how cloudy or opaque the stool sample appears, or how much suspended matter is within the liquid stool sample. The turbidity measurement provides information regarding the content and consistency of the stool and provides an indication of how clear the sample is or is not. The clarity of the stool sample and the turbidity of the stool sample may be used to calculate a clarity score. The clarity score may be numerical scale or a word based scale. For example, the clarity score may be numerical and in a range of numbers such as from 1-5, 1-10 or from 1-100. Where a high score may indicate that the stool sample is completely opaque or not clear and a low score may indicate that the stool sample is completely clear or translucent. A word based scale may include words such as “excellent,” “good,” “fair,” or “poor” where each word on the scale relates to a clarity level of the stool sample. In one implementation, the clarity score may be referred to as a turbidity score. In one implementation, the clarity score is a classification of the clarity of a stool sample.

The assessment at 106 may include a visual inspection of the stool sample. The visual inspection may be analyzed with a human eye, or an image of the stool sample may be captured by a camera or data associated with the stool sample may be captured by a digital data capturing device and then processed and analyzed by a computer executed algorithm or by an artificial intelligence and/or machine learning (AI/ML) algorithm to classify the quality of bowel preparation. The assessment at 106 may include assessing the stool sample with one or more digital data capturing devices including a spectrophotometer, a turbidimeter, or a nephelometer. The assessment at 106 may include viewing with a human eye or recording a photograph or video of a device, such as a Secchi disk, symbol, or image through the stool sample, and then assessing visibility by computer-executed application or by an artificial intelligence and/or machine learning (AI/ML) algorithm to classify the quality of bowel preparation. When referring to the term “Secchi disk,” this disclosure also contemplates images, barcodes, quick response codes, number(s), letter(s), or other marking for visual assessment through the fluid.

The method 100 includes determining at 108 whether the intestine is sufficiently clean for a successful lower gastrointestinal tract procedure with favorable visualization. The determining 108 may be based on the clarity score and may be performed by the AI/ML algorithm or engine. The determining at 108 may be performed by a human eye visualizing a Secchi disk, similar device, or other image that is disposed or printed within the collection device for assessing the stool sample. The determining at 108 may further be performed by a human eye by comparing a clarity of the stool sample against a plurality of exemplary stool clarities and determining which exemplary clarity the stool sample most closely matches. The determination may be compared against an objective measurement to assess whether the sample is clear enough to provide favorable visualization during a procedure.

The determining at 108 may also be performed based on data output by one or more of a spectrophotometer, turbidimeter, or nephelometer. The determining at 108 may be output by an AI/ML engine that has been fed an image of the stool sample and/or data output by one or more of a spectrophotometer, turbidimeter, or nephelometer. The determining at 108 may be performed based on the results of utilizing a Secchi disk, similar device, or other image. The determining at 108 may compare a clarity of the stool sample against a plurality of exemplary stool clarities and determining which exemplary clarity the stool sample most closely matches. The determination may be compared against an objective measurement to assess whether the sample is clear enough to provide favorable visualization during a procedure. The determining at 108 may be based on the clarity score described above.

The lower gastrointestinal tract of the patient may be classified as sufficiently clean to provide favorable visualization to undergo the intended procedure if the stool sample satisfies a threshold clarity or transparency requirement. Such a threshold provides an objective standard by which clarity of the stool sample may be assessed. If the stool sample fails to the threshold for clarity, then the determination at 108 will indicate a likelihood that the patient's lower gastrointestinal tract is not sufficiently clean to undergo a procedure with favorable visualization. Conversely, if the stool sample meets the threshold for clarity, then the determination at 108 will indicate a higher likelihood that the patient's lower gastrointestinal tract is sufficiently clean to undergo a procedure with favorable visualization.

The method 100 may include providing at 110 a determination result to a patient and/or provider. The providing at 110 may include electronically communicating with an account associated with the patient and/or a provider of the patient to indicate whether the patient's intestine is sufficiently clean to undergo a successful lower gastrointestinal tract procedure with favorable visualization.

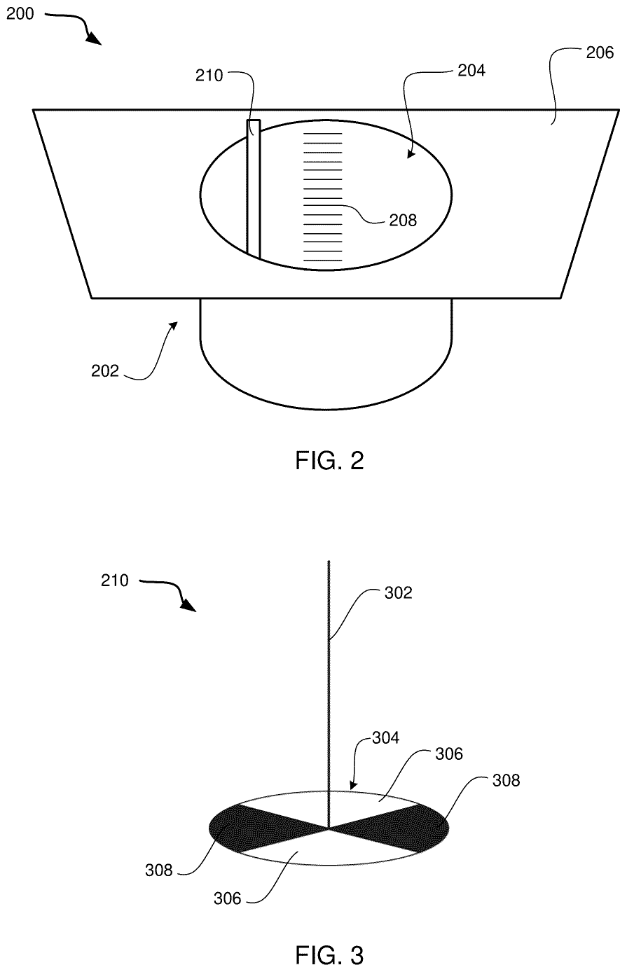

FIG. 2 is a schematic illustration of a system 200 for evaluating whether the lower gastrointestinal tract of a patient is sufficiently prepared to provide favorable visualization during a gastrointestinal procedure. The system 200 may be utilized by a patient and/or a provider to collect a stool sample, assess the clarity or transparency of the stool sample, and make an objective determination of the cleanliness of the patient's lower gastrointestinal tract.

The system 200 includes a specimen collector 202 for receiving a stool sample. The specimen collector 202 may be referred to as a specimen containing device. The exemplary specimen collector 202 illustrated in FIG. 2 is configured to be placed on a toilet or commode to directly catch a stool sample during a bowel movement. However, the specimen collector 202 may alternatively comprise one or more of a bedpan, fecal management system (FMS), toilet bowl, colostomy bag, cup, bag, tube, or another device capable of holding a stool sample that may be analyzed and assessed for clarity and/or turbidity. The specimen collector 202 may comprise a reusable collector constructed from a metal or another material capable of undergoing a sterilization cycle. The specimen collector 202 may comprise a single-use collector constructed from plastic, a flushable material, a biodegradable material, or another suitable material that is disposable.

In some cases, the specimen collector 202 comprises a combination of devices. For example, the specimen collector 202 may comprise a commode collector as illustrated in FIG. 2 and may additionally include a secondary specimen collector 202 such as a cup, tube, or bag. In this case, the stool sample may be directly collected within a commode collector like the device illustrated in FIG. 2, and then the stool sample may be transferred to the secondary specimen collector for testing.

The exemplary specimen collector 202 illustrated in FIG. 2 includes a specimen bowl 204 and a brim 206 attached to the specimen bowl 204. The specimen bowl 204 comprises a plurality of depth markings 208 that are etched into an interior surface of the specimen bowl 204 and/or printed onto the interior surface of the specimen bowl 204. The specimen bowl 204 is configured to be disposed within a bowl of the toilet or commode, and the brim 206 is configured to rest on a rim of the toilet or commode. The brim 206 may be disposed in between the rim of the toilet and the seat of the toilet when the specimen collector 202 is in use. The specimen bowl 204 of the specimen collector 202 is configured to receive a stool specimen of a patient.

The system 200 may include a Secchi disk 210, a similar device, or a similar image for assisting in the determination of clarity of the sample. The Secchi disk 210 may be printed in or otherwise disposed within the specimen bowl 204 as shown in FIG. 2. In some cases, a stool sample may be deposited within the specimen bowl 204, and then a user may lower the Secchi disk 210 into the specimen bowl 204. The markings on the Secchi disk 210 and/or the position of the Secchi disk 210 within the specimen bowl 204 may be utilized to make an objective determination of the clarity or cleanliness of the patient's lower gastrointestinal tract.

FIG. 3 is a perspective view of a Secchi disk 210 that is utilized in connection with the system 200 illustrated in FIG. 2. The system 200 comprises the Secchi disk 210 for measuring the turbidity of a liquid stool sample disposed within the specimen bowl (see 204). The Secchi disk 210 comprises a rod 302 and a disk 304 and may alternatively be printed inside the collection system 200, without the use of a rod. In the exemplary implementation illustrated in FIG. 3, the disk 304 comprises a plurality of bright portions 306 and a plurality of dark portions 308. The plurality of bright portions 306 and the plurality of dark portions 308 make up a “marking” on the disk 304. The marking may include a design as shown in FIG. 3 and may alternatively include any other design, such as an image, barcode, quick response code, number(s), letter(s), or other marking for optical assessment. In some cases, the markings on the disk 304 may comprise letters, numbers, or designs of graduated sizes similar to an optician test for eyesight. The markings may be printed on the disk 304. In some cases, the disk 304 may comprise raised markings.

In some cases, a user is instructed to lower the Secchi disk 210 into a liquid stool sample disposed within a specimen collector (see, e.g., 202). The user may be instructed to lower the Secchi disk 210 into the liquid stool sample only until the markings on the disk 304 are no longer visible. The depth at which the markings on the disk 304 are no longer visible is taken as an objective measurement of the transparency of the liquid stool sample. This measurement may be referred to as the “Secchi depth” and is related to water turbidity. The Secchi depth is reached when the reflectance equals the intensity of light backscattered from the liquid stool sample.

In some cases, a user is instructed to lower the Secchi disk 210 into the liquid stool sample, while capturing an image, a series of images, or a video stream of the process of lowering the Secchi disk 210. The image, series of images, or video stream may be provided to a computer-executed application and/or AI/ML algorithm in real-time, and the computer-executed application and/or AI/ML algorithm may provide a real-time notification indicating at what depth the markings on the disk 304 are determined to no longer be visible. This computer-executed implementation may reduce the incidence of errors by users who do not have prior experience with the Secchi disk 210 method of measuring turbidity of a fluid.

In some cases, a user is instructed to direct a camera towards the Secchi disk 210 disposed within the collection device 200 holding a stool sample (see, e.g., 202). The user may be instructed to capture an image of the Secchi disk 210 through the liquid stool sample. This image may be processed by a computer-executed application and/or AI/ML algorithm to make an objective determination of the resolution or visibility of the markings on the disk 304 when the disk 304 is covered by a certain depth of the stool sample. This visibility determination may be utilized to determine the clarity of the stool sample, and thus determine the cleanliness of the patient's lower gastrointestinal tract.

The system 200 may be implemented with varying types of Secchi disks 210 and may further be implemented with a similar device that does not comprise a Secchi disk 210. For example, the system 200 may be implemented with a coin, chip, sheet, or other device that may be dropped into the specimen bowl 204 with the patient's stool sample. The device may comprise markings that will only be visible or readable if the patient's stool sample is sufficiently clear. The device may be biodegradable or flushable for ease of disposability. The visibility of the device and/or the markings on the device may be utilized to make an objective assessment of whether the patient's lower gastrointestinal tract is sufficiently clean to undergo a gastrointestinal visualization procedure with favorable visualization.

The clarity of the stool sample may be assessed through various visualization and/or optical assessment techniques. One such technique includes the Secchi disk 210 described in connection with FIGS. 2 and 3. However, other optical assessment techniques may be utilized without departing from the scope of the disclosure, including, for example, a Jackson-candle turbidimeter, Tyndall effect, or Chinese turbidity tube. A variety of light sources, images, or designations may be utilized to inspect through the stool sample. These assessment techniques may be utilized with the human eye and/or an image of the assessment technique may be captured with a camera and then processed with a computer-executed algorithm.

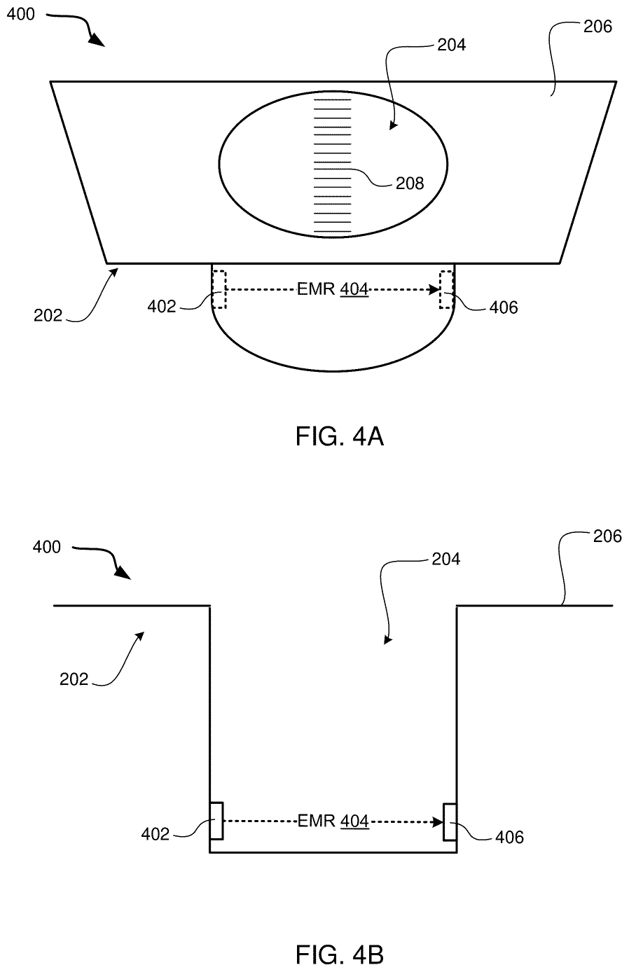

FIGS. 4A and 4B are schematic illustrations of a system 400 for evaluating whether the lower gastrointestinal tract of a patient is sufficiently clean to undergo a successful gastrointestinal procedure with favorable visualization. FIG. 4A is a schematic illustration of a perspective view of the system 400, and FIG. 4B is a schematic illustration of a straight-on cross-sectional side view of the system 400.

The system 400 includes a specimen collector 202 and may specifically include components of the specimen collector 202 first described in connection with FIG. 2. The specimen collector 202 illustrated in FIGS. 4A-4B is exemplary only, and it should be understood that varying types and designs of specimen collectors 202 may be utilized without departing from the scope of the disclosure. For example, the specimen collector 202 may comprise a commode specimen collector as illustrated in FIGS. 4A-4B. Alternatively, the specimen collector 202 may comprise one or more of a bedpan, fecal management system (FMS), toilet bowl, colostomy bag, cup, bag, tube, or another device capable of holding a stool sample. The specimen collector 202 may comprise a reusable collector constructed from metal or another material capable of undergoing a sterilization cycle. The specimen collector 202 may comprise a single-use collector constructed from plastic, a flushable material, a biodegradable material, or another suitable material.

The system 400 includes a light source 402 mounted to a sidewall of the specimen bowl 204. The system 400 additionally includes a spectrophotometer 406 mounted to the sidewall of the specimen bowl 204 such that the spectrophotometer 406 is disposed opposite to the light source 402 such that the stool sample is located between the light source 402 and the spectrophotometer 406. The light source 402 emits electromagnetic radiation (EMR) 404 that transmits through the stool sample and irradiates a photodetector of the spectrophotometer 406. As used herein, the term “photodetector” refers to and intends a range of sensors, including a photocell or an optoelectronic sensor, such as a photodiode. The EMR 404 is scattered by particulates within the stool sample, and this impacts the irradiation of the spectrophotometer 406. The data output by the spectrophotometer 406 may be provided to computer-executed algorithm and/or AI/ML engine configured to determine whether the stool sample is sufficiently clear to indicate that the patient is prepared for a colonoscopy procedure.

In some cases, the light source 402 and the spectrophotometer 406 are not mounted to the specimen collector 202 and are instead separate from the specimen collector 202. In these cases, a user may be instructed to remove a portion of the stool sample from the specimen collector 202 and then evaluate the stool sample with a spectrophotometer 406.

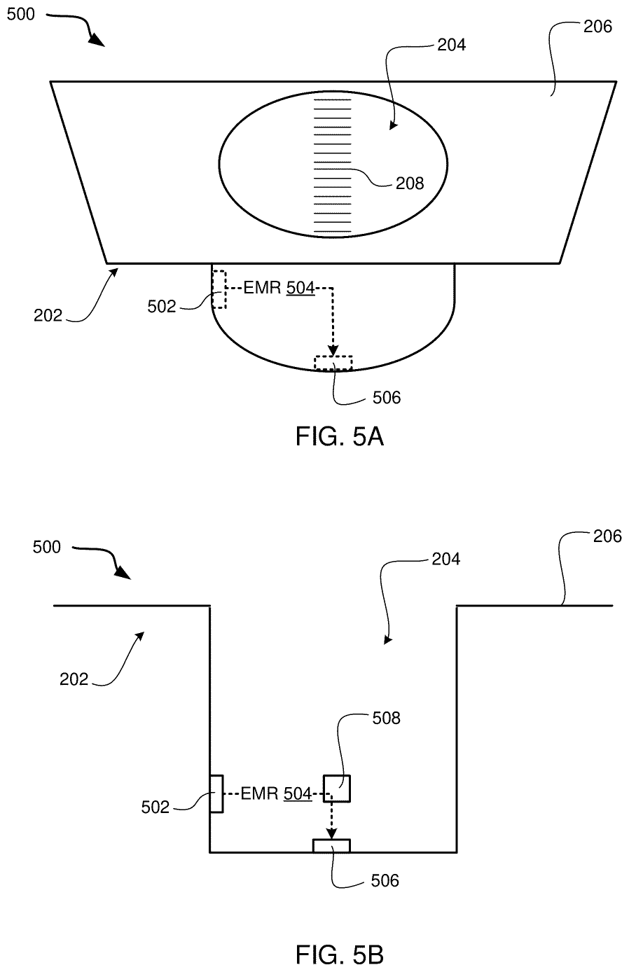

FIGS. 5A and 5B are schematic illustrations of a system 500 for evaluating whether the lower gastrointestinal tract of a patient is sufficiently clean to undergo a successful gastrointestinal visualization procedure with favorable visualization. FIG. 5A is a schematic illustration of a perspective view of the system 500, and FIG. 5B is a schematic illustration of a straight-on cross-sectional side view of the system 500.

The system 500 includes a specimen collector 202 and may specifically include components of the specimen collector 202 first described in connection with FIG. 2. The specimen collector 202 illustrated in FIGS. 5A-5B is exemplary only, and it should be understood that varying types and designs of specimen collectors 202 may be utilized without departing from the scope of the disclosure. For example, the specimen collector 202 may comprise a commode specimen collector as illustrated in FIGS. 5A-5B. Additionally or alternatively, the specimen collector 202 may comprise one or more of a bedpan, fecal management system (FMS), toilet bowl, colostomy bag, cup, bag, tube, or another device capable of holding a stool sample. The specimen collector 202 may comprise a reusable collector constructed from metal or another material capable of undergoing a sterilization cycle. The specimen collector 202 may comprise a single-use collector constructed from plastic, a flushable material, a biodegradable material, or another suitable disposable material.

The system 500 includes a light source 502 mounted to a sidewall of the specimen bowl 204. The system 500 additionally includes a nephelometer 506 mounted to a base of the specimen bowl 204 such that the nephelometer 506 is disposed at substantially a 90° angle relative to the light source 502. The light source 502 emits EMR 504 that transmits through the stool sample and irradiates a photodetector of the nephelometer 506. The system 500 may include an optical element 508 configured to bend the EMR 504 to cause the EMR 504 to irradiate a photodetector of the nephelometer 506. The EMR 504 is scattered by particulates within the stool sample, and this impacts the irradiation of the nephelometer 506. The data output by the nephelometer 506 may be provided to a computer-executed algorithm and/or AI/ML engine configured to determine whether the stool sample is sufficiently clear to indicate that the patient is prepared for a colonoscopy procedure.

In some cases, the light source 502 and the nephelometer 506 are not mounted to the specimen collector 202 and are instead separate from the specimen collector 202. In these cases, a user may be instructed to remove a portion of the stool sample from the specimen collector 202 and then evaluate the stool sample with a nephelometer 506.

The nephelometer 506 may be calibrated according to the Formazin method or another method. The nephelometer 506 may output turbidity measurements expressed in Nephelometric Turbidity Units (NTU), Formazin Nephelometric Units (FNU), and/or Formazin Attenuation Units (FAU). In some cases, numerous stool samples may be assessed with a nephelometer 506, and the nephelometer 506 data outputs may be assessed to determine varying thresholds that define stool sample clarity score as “excellent,” “good,” “fair,” or “poor.” A good quality stool sample would indicate a high likelihood that a patient's intestine is sufficiently clean to undergo a successful gastrointestinal procedure with favorable visualization. Alternatively, a numerical score can be employed to define stool sample clarity score.

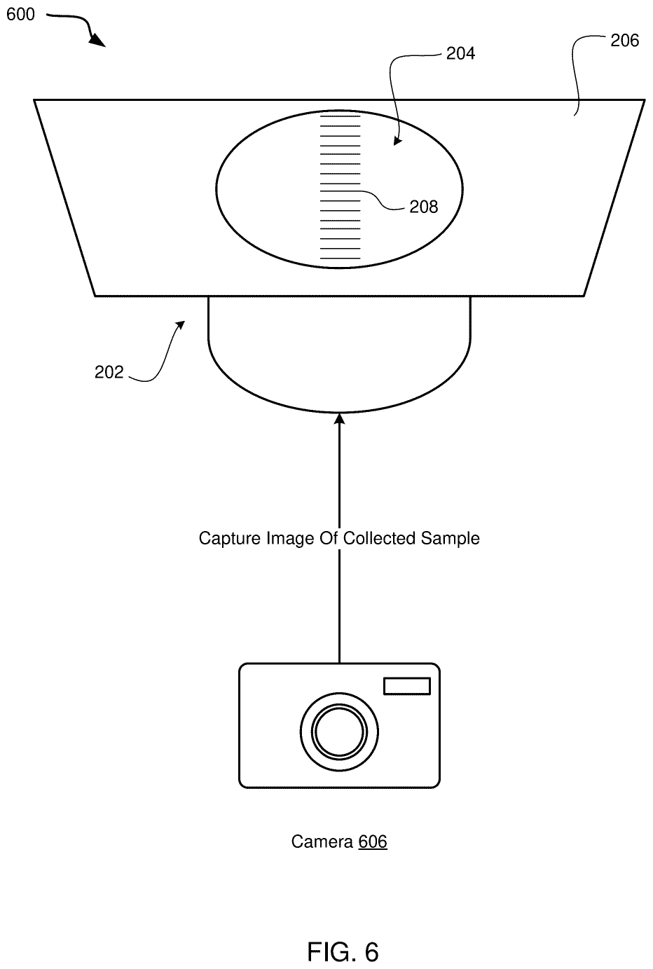

FIG. 6 is a schematic illustration of a system 600 for evaluating whether the lower gastrointestinal tract of a patient is sufficiently clean to undergo a successful gastrointestinal procedure with favorable visualization.

The system 600 includes a specimen collector 202 and may specifically include components of the specimen collector 202 first described in connection with FIG. 2. The specimen collector 202 illustrated in FIG. 6 is exemplary only, and it should be understood that varying types and designs of specimen collectors 202 may be utilized without departing from the scope of the disclosure. For example, the specimen collector 202 may comprise a commode specimen collector as illustrated in FIG. 6. Additionally, or alternatively, the specimen collector 202 may comprise one or more of a bedpan, fecal management system (FMS), toilet bowl, colostomy bag, cup, bag, tube, or another device capable of holding a stool sample. The specimen collector 202 may comprise a reusable collector constructed from metal or another material capable of undergoing a sterilization cycle. The specimen collector 202 may comprise a single-use collector constructed from plastic, a flushable material, a biodegradable material, or another suitable disposable material.

The system 600 further includes a camera 606, which may include a camera 606 integrated within a personal computing device such as a mobile phone or tablet. A user utilizes the system 600 by collecting a liquid stool sample within a specimen bowl 204 and then capturing an image or video stream of the liquid stool sample with the camera 606. The image or video stream of the liquid stool sample may be captured when the stool sample is disposed within the specimen bowl 204 or has been removed from the specimen bowl 204. The image data captured by the camera 606 may be provided to a computer-executed algorithm and/or AI/ML engine to determine whether the stool sample is sufficiently clear to indicate the patient is prepared to undergo a gastrointestinal visualization procedure.

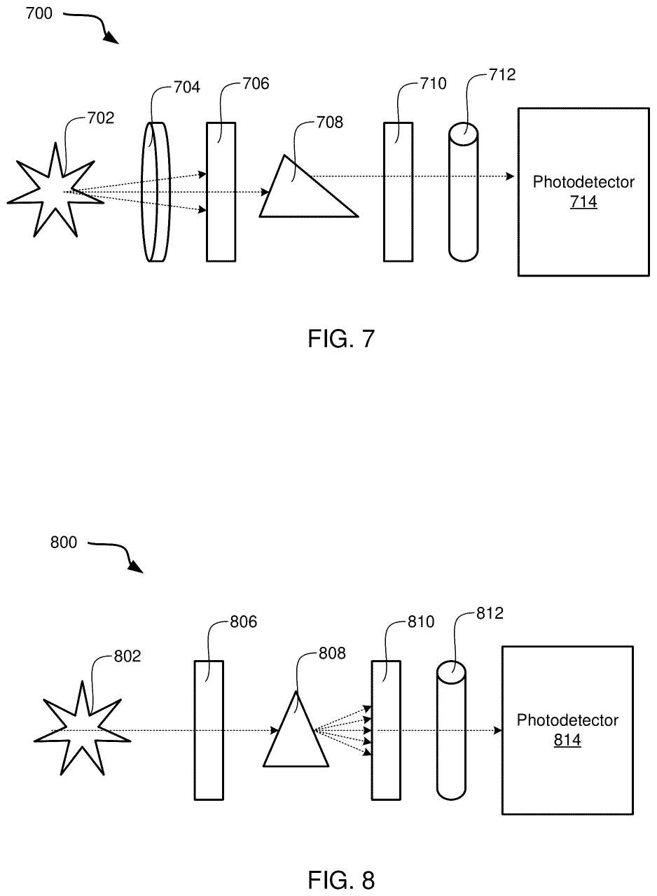

FIG. 7 is a schematic illustration of components of a system 700 for turbidimetry. The system 700 may be integrated within a turbidimeter as described herein. The system 700 includes a light source 702 that emits electromagnetic radiation into a collimating system 704. The electromagnetic radiation 702 is split by the collimating system 704 prior to passing through an entrance slit 706. The electromagnetic radiation passes through a monochromator 708 and then through an exit slit 710 prior to transmitting through the sample 712. The electromagnetic radiation passes transmits through the sample 712 and irradiates a photodetector 714, which may then be objectively analyzed to determine whether the sample is sufficiently clear to indicate the patient is prepared to undergo a gastrointestinal visualization procedure.

FIG. 8 is a schematic illustration of components of a system 800 for spectrophotometry. The system 800 may be integrated within a spectrophotometer as described herein. The system 800 includes a light source 802 that emits electromagnetic radiation into an entrance slit 806. The electromagnetic radiation passes through a dispersion device 808 and then through an exit slit 810 prior to transmitting through the sample 812. The electromagnetic radiation passes transmits through the sample 812 and irradiates a photodetector 814, which may then be objectively analyzed to determine whether the sample is sufficiently clear to indicate the patient is prepared to undergo a gastrointestinal visualization procedure.

The dispersion device 808 may specifically include a monochromator comprising a diffraction grating for producing an analytical spectrum. The diffraction grating of the dispersion device 808 may be movable or fixed.

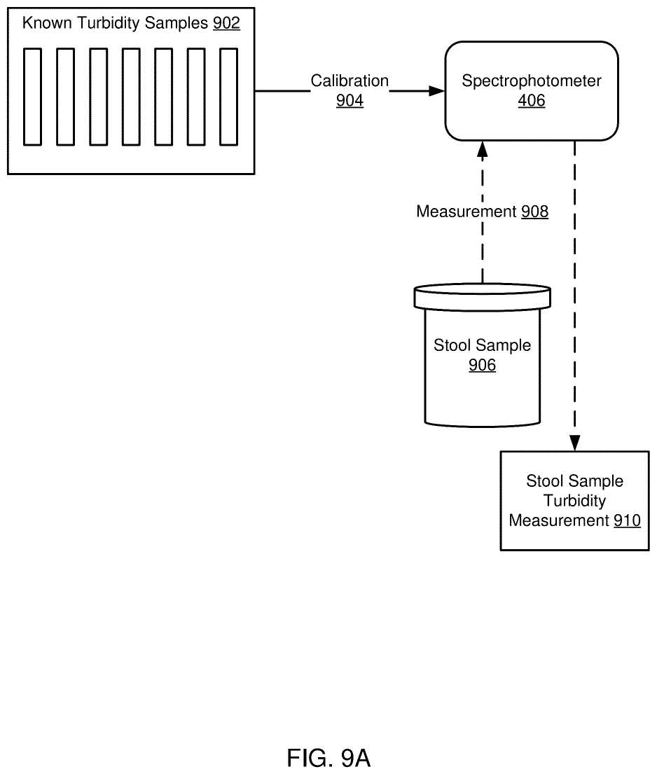

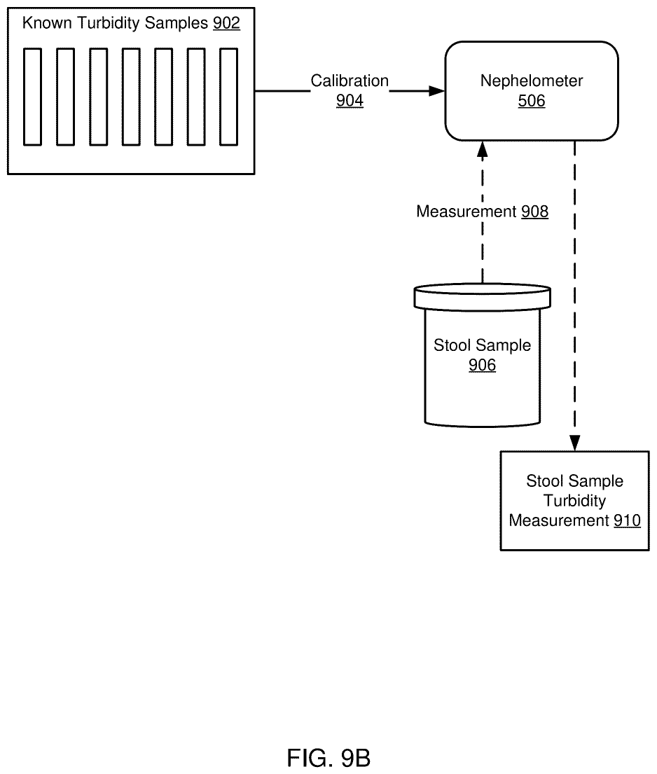

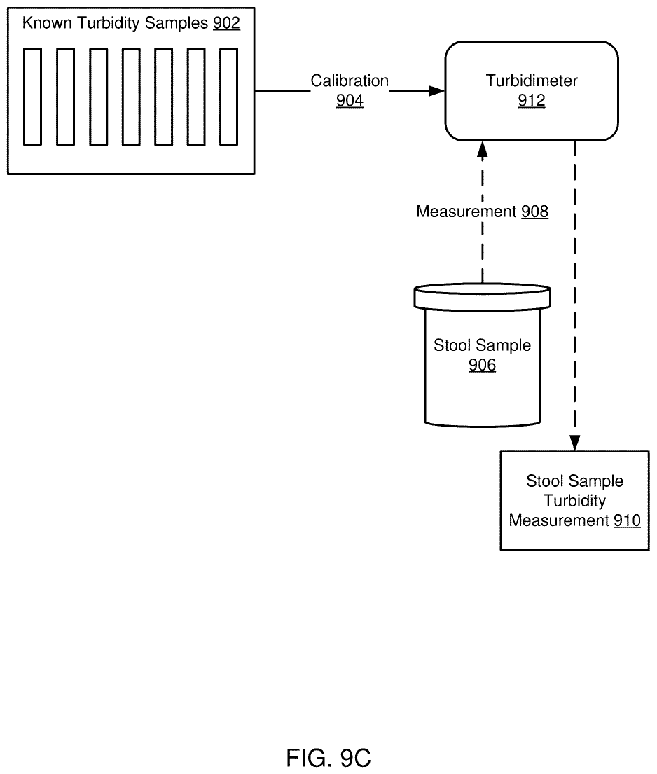

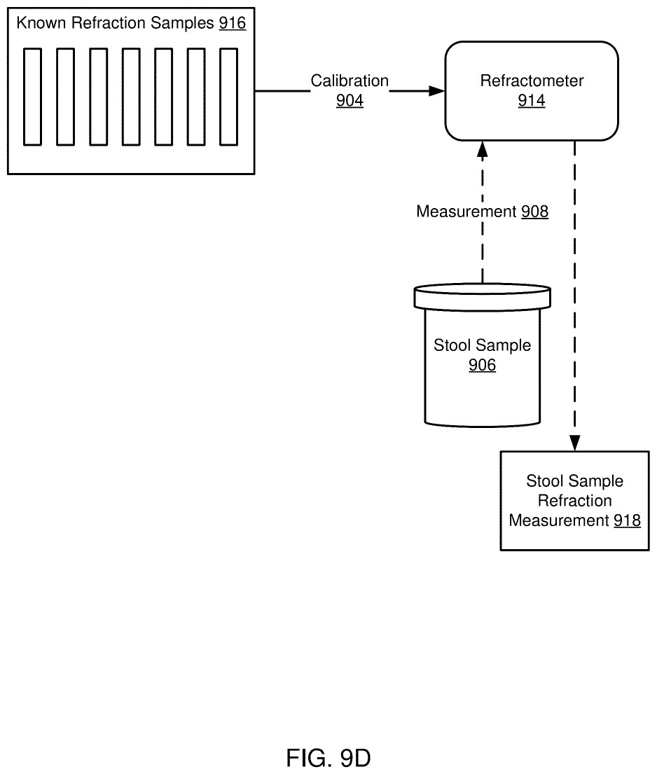

FIGS. 9A-9D are schematic diagrams of a system and process flow for calibrating an assessment device that may be utilized in connection with the systems, methods, and devices described herein. FIG. 9A is a schematic diagram of a system and process flow for calibrating a spectrophotometer 406 to assess the turbidity of a stool sample. FIG. 9B is a schematic diagram of a system and process flow for calibrating a nephelometer 506 to assess turbidity of a stool sample. FIG. 9C is a schematic diagram of a system and process for calibrating a turbidimeter 912 to assess the turbidity of a stool sample. FIG. 9D is a schematic diagram of a system and process for calibrating a refractometer 914 to assess the refraction of light through a stool sample.

As shown in FIG. 9A, a spectrophotometer 406 may be calibrated to assess a stool sample 906 and classify the turbidity of the stool sample 906. The stool sample 906 may be located in the specimen containing device. The process flow includes calibration at 904 of the spectrophotometer 406 by causing the spectrophotometer 406 to measure a plurality of known turbidity samples 902. Each of the known turbidity samples 902 is prepared to comprise a known turbidity value, and the standards of the known turbidity samples 902 are selected to cover the expected range of turbidity in the liquid stool samples 906 to be measured as described herein.

After the spectrophotometer 406 is calibrated with the known turbidity samples 902, the spectrophotometer 406 may be utilized to measure at 908 the turbidity of the stool sample 906. The spectrophotometer 406 outputs the patient sample turbidity measurement 910. The stool sample turbidity measurement 910 may be provided to a patient and/or provider to determine whether the turbidity measurement 910 indicates the stool is sufficiently clear, and thus indicates the patient's lower gastrointestinal tract is likely sufficiently clean to undergo a gastrointestinal visualization procedure with favorable results. Additionally, or alternatively, the stool sample turbidity measurement 910 may be provided to a computer-executed algorithm and/or AI/ML engine configured to determine whether the turbidity is sufficiently low to indicate the lower gastrointestinal tract of the patient is sufficiently prepared for the gastrointestinal visualization procedure.

As shown in FIG. 9B, a nephelometer 506 may be calibrated to assess a stool sample 906 and classify the concentration of suspended particulates (i.e., the turbidity) of the stool sample 906. The stool sample 906 may be located in the specimen containing device. The process flow includes calibration at 904 of the nephelometer 506 by causing the nephelometer 506 to measure a plurality of known turbidity samples 902. Each of the known turbidity samples 902 is prepared to comprise a known turbidity value, and the standards of the known turbidity samples 902 are selected to cover the expected range of turbidity in the liquid stool samples 906 to be measured as described herein.

After the nephelometer 506 is calibrated with the known turbidity samples 902, the nephelometer 506 may be utilized to measure at 908 the turbidity of the stool sample 906. The nephelometer 506 outputs the stool sample turbidity measurement 910. The stool sample turbidity measurement 910 may be provided to a patient and/or provider to determine whether the turbidity measurement 910 indicates the stool is sufficiently clear, and thus indicates the patient's lower gastrointestinal tract is likely sufficiently clean to undergo a gastrointestinal visualization procedure with favorable results. Additionally, or alternatively, the stool sample turbidity measurement 910 may be provided to a computer-executed algorithm and/or AI/ML engine configured to determine whether the turbidity is sufficiently low to indicate the lower gastrointestinal tract of the patient is sufficiently prepared for the gastrointestinal visualization procedure.

As shown in FIG. 9C, a turbidimeter 912 may be calibrated to assess a stool sample 906 and classify the turbidity of the stool sample 906. The stool sample 906 may be located in the specimen containing device. The process flow includes calibration at 904 of the turbidimeter 912 by causing the turbidimeter 912 to measure a plurality of known turbidity samples 902. Each of the known turbidity samples 902 is prepared to comprise a known turbidity value, and the standards of the known turbidity samples 902 are selected to cover the expected range of turbidity in the liquid stool samples 906 to be measured as described herein.

After the turbidimeter 912 is calibrated with the known turbidity samples 902, the turbidimeter 912 may be utilized to measure at 908 the turbidity of the stool sample 906. The turbidimeter 912 outputs the stool sample turbidity measurement 910. The stool sample turbidity measurement 910 may be provided to a patient and/or provider to determine whether the turbidity measurement 910 indicates the stool is sufficiently clear, and thus indicates the patient's lower gastrointestinal tract is likely sufficiently clean to undergo a gastrointestinal visualization procedure with favorable results. Additionally, or alternatively, the stool sample turbidity measurement 910 may be provided to a computer-executed algorithm and/or AI/ML engine configured to determine whether the turbidity is sufficiently low to indicate the lower gastrointestinal tract of the patient is sufficiently prepared for the gastrointestinal visualization procedure.

As shown in FIG. 9D, a refractometer 914 may be calibrated to assess a stool sample 906 and measure the refraction of light through the stool sample 906. The stool sample 906 may be located in the specimen containing device. The refraction of light through the stool sample 906 may be utilized to determine the concentration or purity of the stool sample 906. The process flow illustrated in FIG. 9D includes calibration at 904 of the refractometer 914 by causing the refractometer 914 to measure a plurality of known refraction samples 916. Each of the known refraction samples 916 is prepared to comprise a known refraction value, and the standards of the known refraction sample 916 are selected to cover the expected range of refraction and purity in the liquid stool samples 906 to be measured as described herein.

After the refractometer 914 is calibrated with the known refraction samples 916, the refractometer 914 may be utilized to measure at 908 the refraction of light through the stool sample 906. The refractometer 914 outputs the stool sample refraction measurement 918. The stool sample refraction measurement 918 may be provided to a patient and/or provider to determine whether the refraction measurement 918 indicates the stool is sufficiently clear and pure, and thus indicates the patient's lower gastrointestinal tract is likely sufficiently clean to undergo a gastrointestinal visualization procedure with favorable results. Additionally, or alternatively, the stool sample refraction measurement 918 may be provided to a computer-executed algorithm and/or AI/ML engine configured to determine whether the purity or clarity is sufficiently high to indicate the lower gastrointestinal tract of the patient is sufficiently prepared for the gastrointestinal visualization procedure.

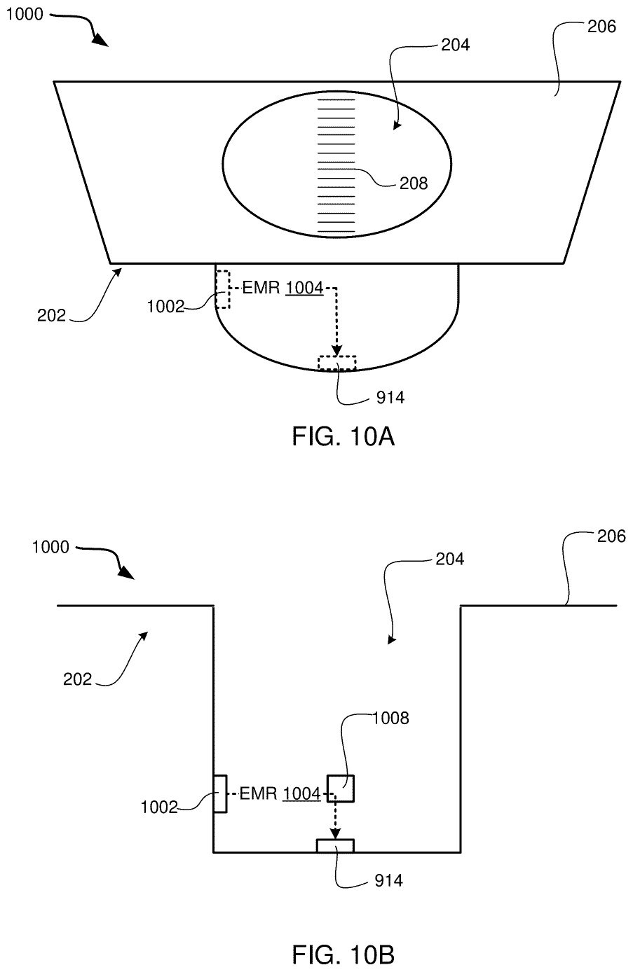

FIGS. 10A and 10B are schematic illustrations of a system 1000 for evaluating whether the lower gastrointestinal tract of a patient is sufficiently clean to undergo a successful gastrointestinal visualization procedure with favorable visualization. FIG. 10A is a schematic illustration of a perspective view of the system 1000, and FIG. 10B is a schematic illustration of a straight-on cross-sectional side view of the system 1000.

The system 1000 includes a specimen collector 202 and may specifically include components of the specimen collector 202 first described in connection with FIG. 2. The specimen collector 202 illustrated in FIGS. 10A-10B is exemplary only, and it should be understood that varying types and designs of specimen collectors 202 may be utilized without departing from the scope of the disclosure. For example, the specimen collector 202 may comprise a commode specimen collector as illustrated in FIGS. 10A-10B. Additionally or alternatively, the specimen collector 202 may comprise one or more of a bedpan, fecal management system (FMS), toilet bowl, colostomy bag, cup, bag, tube, or another device capable of holding a stool sample. The specimen collector 202 may comprise a reusable collector constructed from metal or another material capable of undergoing a sterilization cycle. The specimen collector 202 may comprise a single-use collector constructed from plastic, a flushable material, a biodegradable material, or another suitable disposable material.

The system 1000 includes a light source 1002 mounted to a sidewall of the specimen bowl 204. The system 1000 additionally includes a refractometer 914 mounted to the specimen bowl 204 such that the refractometer 914 is disposed at an advantageous angle relative to the light source 1002. The light source 1002 emits EMR 1004 that transmits through the stool sample and irradiates an optical element 1008 configured to bend the EMR 1004 before measurement. The EMR 1004 is scattered by solutes within the stool sample, and this impacts the irradiation of the refractometer 914. The data output by the refractometer 914 may be provided to a computer-executed algorithm and/or AI/ML engine configured to determine whether the stool sample is sufficiently clear to indicate that the patient is prepared for a colonoscopy procedure.

In some cases, the light source 1002 and the refractometer 914 are not mounted to the specimen collector 202 and are instead separate from the specimen collector 202. In these cases, a user may be instructed to remove a portion of the stool sample from the specimen collector 202 and then evaluate the stool sample with a refractometer 914.

The refractometer 914 may output turbidity measurements expressed by Refractive Index (RI). In some cases, numerous stool samples may be assessed with a refractometer 914, and the refractometer 914 data outputs may be assessed to determine varying thresholds that define stool sample clarity score as “excellent,” “good,” “fair,” or “poor.” A good quality stool sample would indicate a high likelihood that a patient's intestine is sufficiently clean to undergo a successful gastrointestinal procedure with favorable visualization. Alternatively, a numerical score can be employed to define stool sample clarity score.

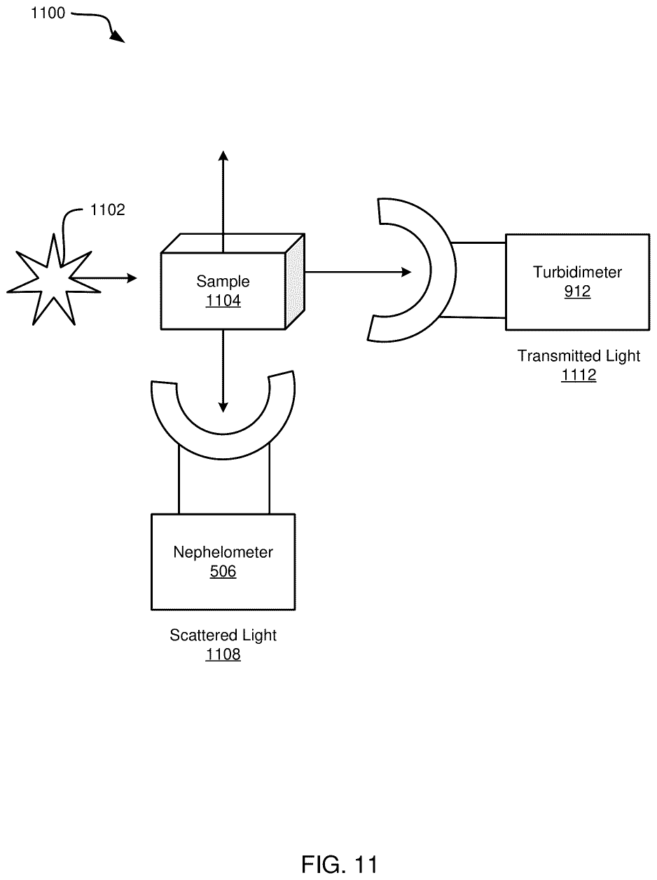

FIG. 11 is a schematic block diagram of a system 1100 for performing simultaneous nephelometry and turbidimetry to determine the turbidity of a sample. The system 1100 may be incorporated into a specimen collector like those described herein.

The system 1100 includes a light source 1102 that emits electromagnetic radiation (EMR) at the sample 1104. The sample 1104 may include a liquid stool sample from a patient. The system 1100 includes a nephelometer 506 device that receives scattered light 1108 that has passed through the sample 1104. The system 1100 includes a turbidimeter 912 that receives transmitted light 1112 that has passed through the sample 1104.

Nephelometer 506 measures the amount of light scattered as the light encounters particles suspended in a solution. Turbidimetry measure the amount of light transmitted as the light encounters particles suspended in a solution. The combination of data outputs from the nephelometer 506 device and the turbidimeter 912 may be utilized to classify the turbidity of a stool sample 1104.

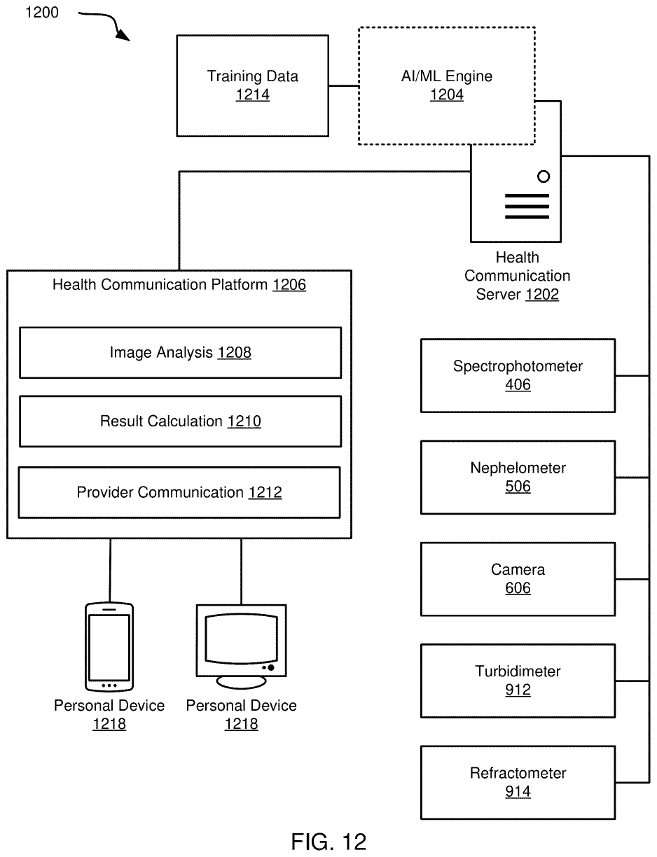

FIG. 12 is a schematic diagram of a system 1200 for data collection, image analysis, and result analysis. The system 1200 may be utilized to perform portions of the method 100 described in connection with FIG. 1. The system 1200 may comprise any of the systems and devices described in connection with any of FIGS. 2-10. The system 1200 may specifically be utilized to assess a stool sample and then determine whether a patient's lower gastrointestinal tract is sufficiently clean to undergo a successful gastrointestinal procedure such as a colonoscopy with favorable visualization. In connection with the system 1200, the camera 606 includes the light sources, photocells, other sensors and other features of a smart-phone or other personal device 1218, as may function through a computer-executed application, and as used for data collection, image analysis, and result analysis. Such computer-executed applications may use light sources, image sensors, photocells, other sensors and other features of a smart-phone or other personal device 1218 to perform the functions of a spectrophotometer, nephelometer, turbidimeter, or refractometer, instead of a stand-alone measuring device.

The system 1200 provides a means for a patient or a provider to receive an objective determination of the cleanliness of the patient's lower gastrointestinal tract prior to a procedure. The system 1200 includes a health communication platform 1206 that comprises a graphical user interface (GUI) accessible with a personal device 1218. A user may provide data to the health communication platform 1206, and then a health communication server 1202 may assess the data and make an objective determination on the cleanliness of the patient's lower gastrointestinal tract. This objective determination may be provided to the user by way of the health communication platform 1206.

The system 1200 includes a health communication server 1202 that includes an artificial intelligence and/or machine learning (AI/ML) engine 1204. The AI/ML engine 1204 is trained upon a set of training data 1214. The health communication server 1202 processes operations for a health communication platform 1206 that is made accessible to users by way of personal devices 1218. The health communication platform 1206 includes numerous modules, and may at least include an image analysis 1208, result calculation 1210, and provider communication 1212 module. The system 1200 includes one or more of a spectrophotometer 406, nephelometer 506, camera 606, turbidimeter 912, or refractometer 914 that is directly or indirectly in communication with the health communication server 1202.

The health communication platform 1206 is made accessible to one or more personal devices 1218 by way of a computer-executed application, web browser, or other means. The health communication server 1202 renders a graphical user interface for the health communication platform 1206. The health communication server 1202 processes data output by one or more of the spectrophotometer 406, nephelometer 506, camera 606, or turbidimeter 100, or personal devices 1218.

The AI/ML engine 1204 is trained to assess input images to objectively evaluate lower gastrointestinal tract cleanliness. The AI/ML engine 1204 is trained on the training data 1214, which comprises a large dataset of input images. The training data 1214 for the AI/ML engine 1204 is utilized to teach the AI/ML engine 1204 to perform specific tasks, including at least analyzing an input image to determine whether a liquid stool sample is sufficiently clear to indicate the patient is prepared for a colonoscopy procedure. The training data 1214 serves as the foundation upon which the AI/ML engine 1204 learns patterns, makes decisions, and generates predictions. The training data 1214 includes structured and unstructured data from various sources, which may include text, images, audio, and sensor data. The training data 1214 specifically includes numerous images of fluids comprising varying degrees of turbidity or clarity. The quality of the training data 1214 is assessed to reduce or eliminate bias and errors in the data. In most cases, data cleaning and data preprocessing is necessary to ensure the quality of the training data 1214. In one implementation, a human may classify or score an image of a stool sample with a clarity score. That image and clarity score may be included in the training data 1214. In one implementation, an image may be taken of a stool sample prior to a procedure such as a colonoscopy. After the colonoscopy has been performed, the health professional who performed the colonoscopy may input a post procedure clarity score of how clear the colon was during the colonoscopy. The post procedure clarity score may be associated with the image of the stool sample taken before the colonoscopy and included as part of the training data 1214. Therefore, training data 1214 may include training data that includes post procedure clarity scores of how well prepared a colon was before the procedure and the AI/ML engine 1204 may be trained based on the post procedure clarity score along with the image associated with the post procedure clarity score of the stool sample before the procedure occurred. Thus the AI/ML engine 1204 may be trained with real world results of how well prepared a colon was based on an image of a stool sample before the procedure took place.

The camera 606 may include a standalone camera or one or more image sensors, light sources, photocells, other sensors, and other features associated with another computing device such as a mobile phone. In some cases, a personal device 1218 comprising a camera communicates with the health communication server 1202 by way of a computer-executed application. The health communication server 1202 executes the application and may communicate directly with the camera 606 to receive images and other data captured by the camera 606.

The spectrophotometer 406 is a device that measures how much electromagnetic radiation (EMR) is absorbed by a substance. The efficiency, resolution, sensitivity, and spectral range of the spectrophotometer 406 is optimized for measuring a liquid stool sample and determining whether a patient's lower gastrointestinal tract is likely sufficiently clean for undergoing a successful colonoscopy procedure.

The spectrophotometer 406 is utilized for electromagnetic spectroscopy, which is the quantitative measurement of the reflection or transmission properties of a material as a function of wavelength. The spectrophotometer 406 quantitatively assesses how much electromagnetic radiation (EMR) is absorbed by colored compounds. Spectrophotometers 406 are typically used to measure the transmittance or reflectance of solutions, transparent or opaque solids, or gases. The spectrophotometer 406 of the system 1200 is tuned to measure the absorption of EMR through a liquid stool sample. The data output by the spectrophotometer 406 is assessed by the health communication server 1202 to determine whether a patient's lower gastrointestinal tract is sufficiently cleaned for a colonoscopy procedure.

The spectrophotometer 406 may include a single-beam spectrophotometer or a double-beam spectrophotometer. The spectrophotometer 406 utilizes photometers that measure the intensity of a light beam at different wavelengths. The spectrophotometer 406 includes a source of electromagnetic radiation (EMR) that emits one or more of ultraviolet EMR, visible EMR, infrared EMR, x-ray EMR, or microwave EMR.

The nephelometer 506 is a device for measuring the concentration of suspended particulates in a fluid. The nephelometer 506 measures suspended particulates by employing a source beam that emits EMR and a photodetector to one side of the source beam. In many cases, the photodetector is oriented substantially perpendicular to the source beam. The nephelometer 506 relies on the principle that particle density is a function of the light reflected into the photodetector from the particles. The nephelometer 506 may be calibrated to a known particulate, and then environmental factors (i.e., k-factors) may be used to compensate lighter or darker colored dusts accordingly.

The camera 606 may be utilized to capture one or more images of a patient stool sample. These images may be processed by the AI/ML engine 1204 to objectively determine whether the patient is prepared for a colonoscopy procedure. The camera 606 may be associated with a personal device 1218. For example, the camera 606 may include one or more image sensors attached to and associated with a personal device 1218 such as a mobile phone. The camera 606 may include any suitable computing device capable of communicating directly or indirectly with the health communication server 1202.

The turbidimeter 912 is a device for measuring the turbidity of a liquid, which relates to the loss of intensity of transmitted EMR due to the scattering effect of particles suspended within the liquid. Turbidity is the cloudiness or haziness of a fluid caused by a number of individual particles that are generally invisible to the naked eye. The measurement of turbidity can be an important test for assessing water clarity and water quality. Fluids can contain suspended solid matter comprising particles of varied sizes. The data output by the turbidimeter 912 is processed by the health communication server 1202 and/or the turbidimeter 912 itself to calculate the turbidity of a liquid stool sample. If the liquid stool sample has very low turbidity, then the patient's lower gastrointestinal tract is likely sufficiently clean for a colonoscopy procedure with favorable visualization.

The turbidimeter 912 includes a light source that emits EMR through a filter to create an emission of a known wavelength. The filtered EMR is transmitted through a sample comprising a solution. The turbidimeter 912 includes a photoelectric cell that accumulates the filtered EMR that has transmitted through the solution. The turbidimeter 912 outputs data indicating an amount of EMR that was accumulated by the photoelectric cell. The health communication server 1202 and/or the turbidimeter 912 itself processes the data to calculate the turbidity of the solution. The turbidity of the solution may be measured according to the Nephelometric Turbidity Unit (NTU).

The refractometer 914 is an optical instrument used to measure the refractive index of a substance, such as a stool sample. The refractometer 914 may be utilized to calculate the concentration of solutes within a solution. The refractometer 914 functions on the principle that light changes directions, or refracts, when it passes from one medium into another, such as from air into the water solvent of a stool sample. The refractometer 914 may comprise one or more of a handheld refractometer, digital refractometer, or Abbe refractometer.

The personal device 1218 is any personal computing device that can communicate with the health communication server 1202. The personal device 1218 may include a smart phone, a tablet, a laptop, a personal computer, virtual or augmented reality device, and so forth. The personal devices 1218 may communicate with the health communication server 1202 by way of a local area network (LAN) connection, a wide area network (WAN) connection, or another network connection.

The health communication platform 1206 may be rendered on an application that is run on a personal device 1218, such as a mobile phone or other personal computer. The health communication platform 1206 may be rendered on a web browser that is accessible by way of a personal device 1218 connected to the Internet. The health communication platform 1206 comprises functionality for at least performing image analysis 1208, executing result calculation 1210, and facility provider communication 1212 between a patient and a healthcare provider.

The image analysis 1208 is typically executed by the AI/ML engine 1204. A user may utilize the camera 606 to capture an image of a patient stool. The image may then be provided to the health communication server 1202 by way of the health communication platform 1206. The AI/ML engine 1204, which is trained using the training data 1214, then processes the image and provides an objective classification of the cloudiness or the patient's stool. The module for result calculation 1210 is utilized to determine whether the patient is prepared for a colonoscopy procedure based on the image of the patient's stool.

The provider communication 1212 enables a user to provide one or more of an input image or a result calculation 1210 to a patient's healthcare provider. This assessment will inform the healthcare provider of whether the patient's lower gastrointestinal tract is likely sufficiently clean for a colonoscopy procedure with favorable visualization. The healthcare provider and patient (or other authorized party) may communicate bidirectionally through the health communication platform 1206.

In some cases, the system 1200 may include systems or devices for assessing the electrical conductivity and/or electromagnetic flow of a stool system. The data output by the systems or devices may be assessed by the health communication server 1202 to classify a clarity or quality of the stool sample. In these cases, an electrode may be disposed within a specimen collector for collecting the stool sample.

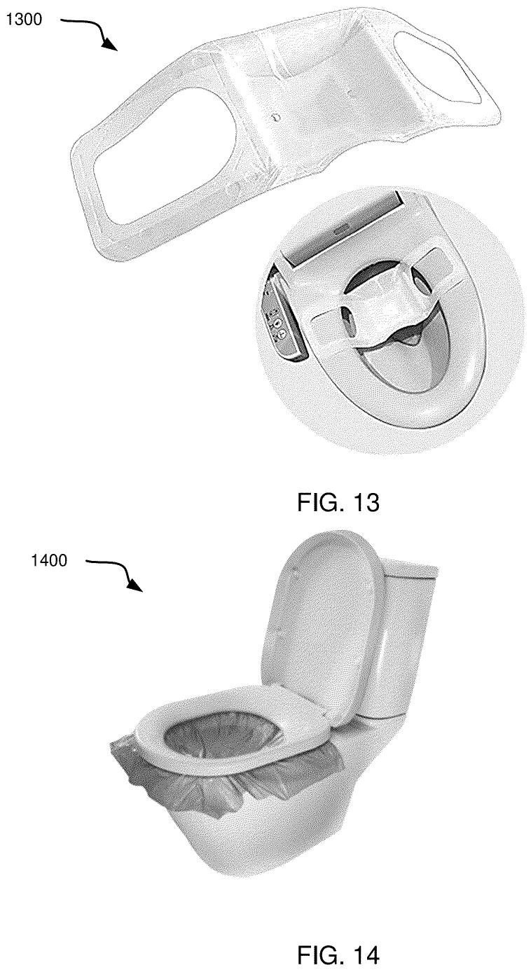

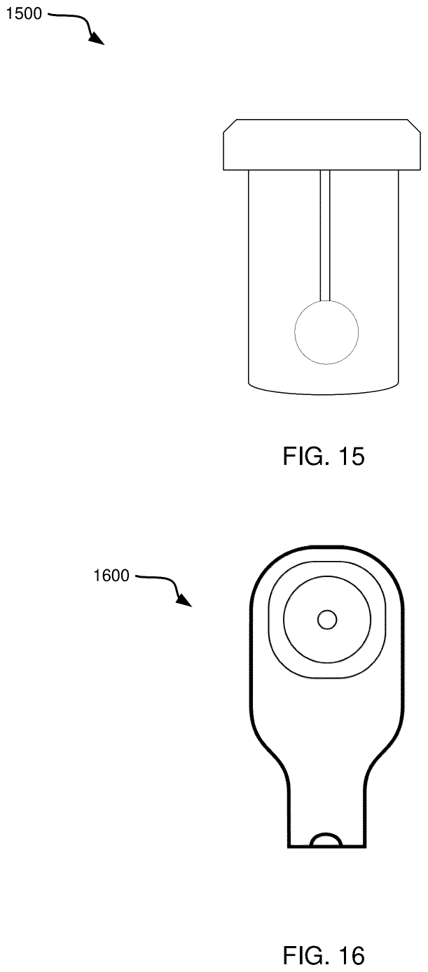

FIGS. 13-16 are images of potential specimen collectors that may be utilized in connection with the systems, methods, and devices described herein. FIG. 13 is an image of a specimen collector 1300 that comprises a flushable or biodegradable commode collection device. FIG. 14 is an image of a specimen collector 1400 that comprises a single-use flushable or biodegradable commode collection device. FIG. 15 is an image of a specimen collector 1500 that comprises a cup or test tube for receiving a stool sample. The specimen collector 1500 may be used as a secondary specimen collector after the stool sample is disposed within commode collector or another device. FIG. 16 is an image of a specimen collector 1600 that comprises a colostomy bag.

Any of the specimen collectors 1300, 1400, 1500, 1600 illustrated in FIGS. 13-16 may be used in connection with any of the systems, methods, or devices described herein. The systems, methods, and devices described herein may be implemented in connection with one or more of a bedpan, fecal management system (FMS), toilet bowl, colostomy bag, biodegradable commode collection device, flushable commode collection device, non-biodegradable commode collection device, stool sample collection cup, stool sample collection tube, and so forth.

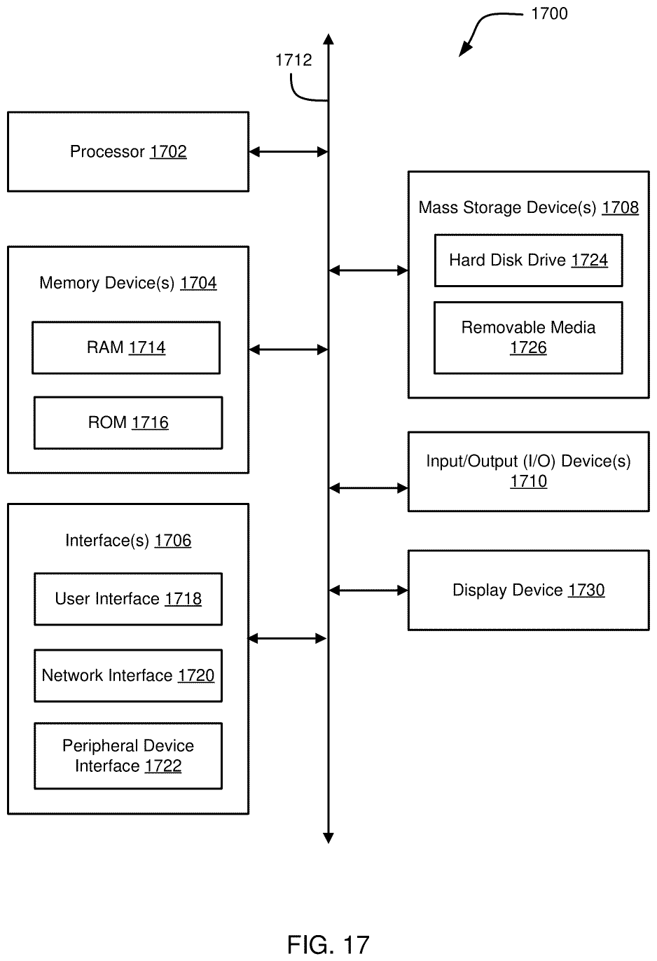

Referring now to FIG. 17, a block diagram of an example computing device 1700 is illustrated. Computing device 1700 may be used to perform various procedures, such as those discussed herein including method 100. Computing device 1700 can perform various monitoring functions as discussed herein, and can execute one or more application programs, such as the application programs or functionality described herein. Computing device 1700 may be used as a standalone device or in conjunction with other computing devices described herein for the AI/ML engine, such as the AI/ML engine 1204 of FIG. 12, the AI/ML network, and/or the neural network described herein. Computing device 1700 can be any of a wide variety of computing devices, such as a desktop computer, in-dash computer, vehicle control system, a notebook computer, a server computer, a handheld computer, tablet computer and the like. The computing device 1700 may also represent personal device 1218 of FIG. 12.

Computing device 1700 includes one or more processor(s) 1712, one or more memory device(s) 1704, one or more interface(s) 1706, one or more mass storage device(s) 1708, one or more Input/output (I/O) device(s) 1710, and a display device 1730 all of which are coupled to a bus 1712. Processor(s) 1712 include one or more processors or controllers that execute instructions stored in memory device(s) 1704 and/or mass storage device(s) 1708. Processor(s) 1712 may also include diverse types of computer-readable media, such as cache memory.

Memory device(s) 1704 include various computer-readable media, such as volatile memory (e.g., random access memory (RAM) 1714) and/or nonvolatile memory (e.g., read-only memory (ROM) 1716). Memory device(s) 1704 may also include rewritable ROM, such as Flash memory.

Mass storage device(s) 1708 include various computer readable media, such as magnetic tapes, magnetic disks, optical disks, solid-state memory (e.g., Flash memory), and so forth. As shown in FIG. 17, a particular mass storage device 1708 is a hard disk drive 1724. Various drives may also be included in mass storage device(s) 1708 to enable reading from and/or writing to the various computer readable media. Mass storage device(s) 1708 include removable media 1726 and/or non-removable media.

I/O device(s) 1710 include various devices that allow data and/or other information to be input to or retrieved from computing device 1700. Example I/O device(s) 1710 include cursor control devices, keyboards, keypads, microphones, monitors, touchscreen devices, or other display devices, speakers, printers, network interface cards, modems, and the like.

Display device 1730 includes any type of device capable of displaying information to one or more users of computing device 1700. Examples of display device 1730 include a monitor, display terminal, video projection device, and the like.

Interface(s) 1706 include various interfaces that allow computing device 1700 to interact with other systems, devices, or computing environments. Example interface(s) 1706 may include any number of different network interfaces 1720, such as interfaces to local area networks (LANs), wide area networks (WANs), wireless networks, and the Internet. Other interface(s) include user interface 1718 and peripheral device interface 1722. The interface(s) 1706 may also include one or more user interface elements 1718. The interface(s) 1706 may also include one or more peripheral interfaces such as interfaces for printers, pointing devices (mice, track pad, or any suitable user interface now known to those of ordinary skill in the field, or later discovered), keyboards, and the like.

Bus 1712 allows processor(s) 1712, memory device(s) 1704, interface(s) 1706, mass storage device(s) 1708, and I/O device(s) 1710 to communicate with one another, as well as other devices or components coupled to bus 1712. Bus 1712 represents one or more of several types of bus structures, such as a system bus, PCI bus, IEEE bus, USB bus, and so forth.

For purposes of illustration, programs and other executable program components are shown herein as discrete blocks, although it is understood that such programs and components may reside at various times in different storage components of computing device 1800 and are executed by processor(s) 1712. Alternatively, the systems and procedures described herein can be implemented in hardware, or a combination of hardware, software, and/or firmware. For example, one or more application specific integrated circuits (ASICs) can be programmed to conduct one or more of the systems and procedures described herein. As used herein, the terms “module” or “component” are intended to convey the implementation apparatus for accomplishing a process, such as by hardware, or a combination of hardware, software, and/or firmware, for the purposes of performing all or parts of operations disclosed herein. The terms “module” or “component” are intended to convey independent in how the modules, components, or their functionality or hardware may be implemented in different embodiments.

Unless defined otherwise, all technical and scientific terms used herein have the same meanings as commonly understood by one of ordinary skill in the art to which this disclosure pertains and belongs.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

### EXAMPLES

The following examples pertain to further embodiments.

Example 1 is a method. The method includes collecting a stool sample from a patient. The method includes assessing the collected stool sample against a threshold to determine whether the intestine of the patient is sufficiently clean for the patient to undergo a lower gastrointestinal tract procedure with favorable visualization.

Example 2 is a method as in Example 1, wherein assessing the stool sample comprises capturing an image of the stool sample.

Example 3 is a method as in any of Examples 1-2, wherein assessing the stool sample further comprises providing the image to an artificial intelligence and/or machine learning algorithm configured to classify a clarity of the stool sample based on the image.

Example 4 is a method as in any of Examples 1-3, wherein assessing the stool sample further comprises providing the image to an artificial intelligence and/or machine learning algorithm configured to classify a turbidity of the stool sample based on the image.

Example 5 is a method as in any of Examples 1-4, wherein collecting the stool sample comprises collecting the stool sample within a specimen collector comprising a bowl.

Example 6 is a method as in any of Examples 1-5, further comprising the assessment of a Secchi disk, printed in or lowered into the specimen bowl, such that the Secchi disk is disposed underneath a depth of the stool sample.

Example 7 is a method as in any of Examples 1-6, further comprising determining the depth of the stool sample disposed above the Secchi disk.

Example 8 is a method as in any of Examples 1-7, wherein determining wherein the intestine of the patient is sufficiently clean comprises determining at least in part based on the depth of the stool sample disposed above the Secchi disk when a marking on the Secchi disk is either no longer visible or exhibits a certain predetermined resolution.

Example 9 is a method as in any of Examples 1-8, further comprising capturing an image of the Secchi disk disposed within the specimen bowl, and wherein determining whether the intestine of the patient is sufficiently clean comprises receiving an output from an AI/ML engine trained to assess the image and classify a clarity of the stool sample.

Example 10 is a method as in any of Examples 1-9, further comprising measuring a clarity of the stool sample with a spectrophotometer.

Example 11 is a method as in any of Examples 1-10, further comprising measuring a clarity of the stool sample with a nephelometer.

Example 12 is a method as in any of Examples 1-11, further comprising measuring a clarity of the stool sample with a turbidimeter.

Example 13 is a method as in any of Examples 1-12, further comprising measuring a clarity of the stool sample with one or more of a spectrophotometer, nephelometer, or turbidimeter, and wherein measuring the clarity comprises: emitting electromagnetic radiation through the stool sample such that the electromagnetic radiation is transmitted through the stool sample; and irradiating a photodetector of one or more of the spectrophotometer, nephelometer, or turbidimeter with the electromagnetic radiation that is transmitted through the stool sample.

Example 14 is a method as in any of Examples 1-12, further comprising measuring a clarity of the stool sample with a refractometer.

Example 15 is a method as in any of Examples 1-13, further comprising measuring a clarity of the stool sample with refractometer, and wherein measuring the clarity comprises: emitting electromagnetic radiation through the stool sample such that the electromagnetic radiation is transmitted through the stool sample and through a prism or similar refracting device; and assessing the bend or refraction of EMR that is transmitted through the stool sample.

Example 16 is a method. The method includes receiving an input image depicting a stool sample. The method includes providing the input image to a neural network trained to identify the stool sample and predict a turbidity of the stool sample. The method includes determining whether a patient is sufficiently prepared for a colonoscopy procedure based on the turbidity of the stool sample as determined by the neural network.

Example 17 is a method. The method includes receiving sensor data output by a turbidimeter, wherein the sensor data is associated with a stool sample. The method includes calculating a turbidity of the stool sample based on the sensor data. The method includes determining whether a patient is sufficiently prepared for a colonoscopy procedure based on the turbidity of the stool sample.

Example 18 is a method. The method includes receiving sensor data output by a spectrophotometer, wherein the sensor data is associated with a stool sample. The method includes calculating a turbidity of the stool sample based on the sensor data. The method includes determining whether a patient is sufficiently prepared for a colonoscopy procedure based on the turbidity of the stool sample.

Example 19 is a method. The method includes receiving a data input comprising a Secchi depth or visual reading from within the stool sample, wherein the Secchi depth or reading indicates a depth measurement wherein a Secchi disk is no longer visible or exhibits a given resolution within the stool sample. The method includes calculating a turbidity of the stool sample based at least in part on the Secchi depth or visual reading. The method includes determining whether a patient is sufficiently prepared for a colonoscopy procedure based on the turbidity of the stool sample.

Example 20 is a system. The system includes a specimen collector comprising a specimen bowl. The system includes a light source mounted to the specimen bowl. The system includes a spectrophotometer mounted to the specimen bowl.

Example 21 is a system. The system includes a specimen collector comprising a specimen bowl. The system includes a light source mounted to the specimen bowl. The system includes a turbidimeter mounted to the specimen bowl.

Example 22 is a system. The system includes a specimen collector comprising a specimen bowl. The system includes a light source mounted to the specimen bowl. The system includes a nephelometer mounted to the specimen bowl.

Example 23 is a system. The system includes a specimen collector comprising a specimen bowl. The system includes a light source mounted to a sidewall of the specimen bowl. The system includes a refractometer mounted to the specimen bowl.

Example 24 is a system. The system includes a specimen containing device for containing a stool sample from a patient. The system includes a digital data capturing device for capturing data from the stool sample in the specimen containing device. The system includes a processor and memory for calculating a clarity score associated with the stool sample based on the data to determine whether the intestine of the patient is sufficiently clean for the patient to undergo a lower gastrointestinal tract procedure with favorable visualization.

Example 25 is a system as in Example 24, wherein the digital data capturing device is an image capturing device for capturing an image and the data is an image.

Example 26 is a system as in any of Examples 24-25, wherein the digital data capturing device is a spectrophotometer.

Example 27 is a system as in any of Examples 24-26, wherein the digital data capturing device is a nephelometer.

Example 28 is a system as in any of Examples 24-27, wherein the digital data capturing device is a refractometer.

Example 29 is a system as in any of Examples 24-28, wherein the digital data capturing device is a turbidimeter.

Example 30 is a system as in any of Examples 24-29, further comprising: a Secchi disk, wherein the Secchi disk is lowered into the specimen containing device after the stool sample is collected or the Secchi disk is integrated with the specimen containing device such that the Secchi disk is disposed underneath a depth of the stool sample; wherein the calculating the clarity score is based on a depth of the stool sample disposed above the Secchi disk.

Example 31 is a system as in any of Examples 24-30, further comprising: a light source configured to emit electromagnetic radiation at the stool sample, wherein a portion of the electromagnetic radiation is transmitted through the stool sample and is collected by the digital data capturing device.

Example 32 is a system as in any of Examples 24-31, wherein clarity score is calculated at least in part by an artificial intelligence and/or machine learning algorithm configured to classify a clarity of the stool sample based on the data.

Example 33 is a system as in any of Examples 24-32, wherein the specimen containing device is a commode specimen collector, a disposable stool collection bag, a stool sample container, a bedpan, a fecal management system (FMS), a toilet bowl, a colostomy bag, and/or a bowl.

Example 34 is a method. The method includes collecting a stool sample from a patient via a specimen containing device. The method includes analyzing the stool sample via a digital data capturing device. The method includes collecting data, from the digital data capturing device, based on the analyzing the stool sample. The method includes calculating a clarity score associated with the stool sample based on the data to determine whether the intestine of the patient is sufficiently clean for the patient to undergo a lower gastrointestinal tract procedure with favorable visualization.

Example 35 is a method as in Example 34, wherein the calculating the clarity score further comprises assessing the stool sample against a threshold.

Example 36 is a method as in any of Examples 34-35, wherein the analyzing the stool sample comprises capturing an image of the stool sample.

Example 37 is a method as in any of Examples 34-36, wherein the analyzing the stool sample further comprises providing the image to an artificial intelligence and/or machine learning algorithm configured to classify a clarity of the stool sample based on the image.

Example 38 is a method as in any of Examples 34-37, wherein determining wherein the intestine of the patient is sufficiently clean comprises determining at least in part based on the depth of the stool sample disposed above a Secchi disk when a marking on the Secchi disk is either no longer visible or exhibits a certain resolution.

Example 39 is a method as in any of Examples 34-38, wherein the digital data capturing device is a spectrophotometer.

Example 40 is a method as in any of Examples 34-39, wherein the digital data capturing device is a nephelometer.

Example 41 is a method as in any of Examples 34-40, wherein the digital data capturing device is a refractometer.

Example 42 is a method as in any of Examples 34-41, wherein the digital data capturing device is a turbidimeter.

Example 43 is a method as in any of Examples 34-42, further comprising: emitting electromagnetic radiation through the stool sample from a light source such that a portion of the electromagnetic radiation is transmitted through the stool sample; and collecting the portion of the electromagnetic radiation that is emitted through the stool sample via the digital data capturing device.

Example 44 is a method. The method includes receiving a digital image of a stool sample from a patient after the patient has undergone a bowel preparation procedure prior to a lower gastrointestinal tract procedure. The method includes assessing the digital image of the stool sample for clarity. The method includes calculating a clarity score based on the clarity of the stool sample in the digital image to determine whether the intestine of the patient is sufficiently clean for the patient to undergo the lower gastrointestinal tract procedure with favorable visualization. The method includes providing the clarity score to the patient or a health care provider.

Example 45 is a method as in Example 44, wherein the calculating the clarity score is performed via an artificial intelligence and/or machine learning engine.

Example 46 is a method as in any of Examples 44-45, wherein the an artificial intelligence and/or machine learning engine is trained based on post procedure clarity scores of patients that have undergone the prior bowel preparation and the lower gastrointestinal tract procedure.

Example 47 is a method as in any of Examples 44-46, further comprising: generating a recommendation, via an artificial intelligence and/or machine learning engine, regarding whether the lower gastrointestinal tract procedure is likely to have a favorable visualization based on the clarity score.

Example 48 is a method as in any of Examples 44-47, further comprising: calculating a clarity score associated with the stool sample based on the data to determine whether the intestine of the patient is sufficiently clean for the patient to undergo the lower gastrointestinal tract procedure with favorable visualization.

Example 49 is a method as in any of Examples 44-48, wherein the clarity score is based at least in part on a turbidity score of the stool sample.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the claims, if any.

What is claimed is:

1. A system comprising:
a specimen containing device for containing a stool sample from a patient;
a digital data capturing device for capturing data from the stool sample in the specimen containing device; and
a processor and memory for calculating a clarity score associated with the stool sample based on the data to determine whether the intestine of the patient is sufficiently clean for the patient to undergo a lower gastrointestinal tract procedure with favorable visualization.

2. The system of claim 1, wherein the digital data capturing device is an image capturing device for capturing an image and the data is an image.

3. The system of claim 1, wherein the digital data capturing device is a spectrophotometer.

4. The system of claim 1, wherein the digital data capturing device is a nephelometer.

5. The system of claim 1, wherein the digital data capturing device is a refractometer.

6. The system of claim 1, wherein the digital data capturing device is a turbidimeter.

7. The system of claim 1, further comprising:
a Secchi disk, wherein the Secchi disk is lowered into the specimen containing device after the stool sample is collected or the Secchi disk is integrated with the specimen containing device such that the Secchi disk is disposed underneath a depth of the stool sample;
wherein the calculating the clarity score is based on a depth of the stool sample disposed above the Secchi disk.

8. The system of claim 1, further comprising:
a light source configured to emit electromagnetic radiation at the stool sample, wherein a portion of the electromagnetic radiation is transmitted through the stool sample and is collected by the digital data capturing device.

9. The system of claim 1, wherein the clarity score is calculated at least in part by an artificial intelligence and/or machine learning algorithm configured to classify a clarity of the stool sample based on the data.

10. The system of claim 1, wherein the specimen containing device is a commode specimen collector, a disposable stool collection bag, a stool sample container, a bedpan, a fecal management system (FMS), a toilet bowl, a colostomy bag, and/or a bowl.

11. A method comprising:
collecting a stool sample from a patient via a specimen containing device;
analyzing the stool sample via a digital data capturing device;
collecting data, from the digital data capturing device, based on the analyzing the stool sample; and
calculating a clarity score associated with the stool sample based on the data to determine whether the intestine of the patient is sufficiently clean for the patient to undergo a lower gastrointestinal tract procedure with favorable visualization.

12. The method of claim 11, wherein the analyzing the stool sample comprises capturing an image of the stool sample; and
wherein the analyzing the stool sample further comprises providing the image to an artificial intelligence and/or machine learning algorithm configured to classify a clarity of the stool sample based on the image.

13. The method of claim 11, wherein the determining the intestine of the patient is sufficiently clean comprises determining at least in part based on the depth of the stool sample disposed above a Secchi disk when a marking on the Secchi disk is either no longer visible or exhibits a certain resolution.

14. The method of claim 11, wherein the digital data capturing device is a spectrophotometer, a nephelometer, a refractometer, and/or a turbidimeter.

15. A method comprising:
receiving a digital image of a stool sample from a patient after the patient has undergone a bowel preparation procedure prior to a lower gastrointestinal tract procedure;
assessing the digital image of the stool sample for clarity;
calculating a clarity score based on the clarity of the stool sample in the digital image to determine whether the intestine of the patient is sufficiently clean for the patient to undergo the lower gastrointestinal tract procedure with favorable visualization; and
providing the clarity score to the patient or a health care provider.

16. The method of claim 15, wherein the calculating the clarity score is performed via an artificial intelligence and/or machine learning engine.

17. The method of claim 15, wherein the an artificial intelligence and/or machine learning engine is trained based on post procedure clarity scores of patients that have undergone the prior bowel preparation and the lower gastrointestinal tract procedure.

18. The method of claim 15, further comprising:
generating a recommendation, via an artificial intelligence and/or machine learning engine, regarding whether the lower gastrointestinal tract procedure is likely to have a favorable visualization based on the clarity score.

19. The method of claim 15, further comprising:
calculating a clarity score associated with the stool sample based on the data to determine whether the intestine of the patient is sufficiently clean for the patient to undergo the lower gastrointestinal tract procedure with favorable visualization.

20. The method of claim 15, wherein the clarity score is based at least in part on a turbidity score of the stool sample.
